SEQUENCE LISTING (44 sequences)

SEQ ID NO: 1 - DNA, 18 bp, Artificial
  23CAT - GR1 Forward Primer
gccgcctccatcatgtgt

SEQ ID NO: 2 - DNA, 17 bp, artificial
  23CAT - GR1 Reverse Primer
ggcgcgaagcacgtctt

SEQ ID NO: 3 - DNA, 17 bp, Artificial
  23CAT - Gr1 Probe
cttctacctcgaaacct

SEQ ID NO: 4 - DNA, 18 bp, Artificial
  NapD - Gr1a Forward Primer
tgggtgacgctgcttggt

SEQ ID NO: 5 - DNA, 18 bp, Artificial
  NapD - Gr1a Reverse Primer
tctaaaccgccggaatgc

SEQ ID NO: 6 - DNA, 17 bp, Artificial
  NapD - Gr1a probe
cctggaacctatgttca

SEQ ID NO: 7 - DNA, 18 bp, Artificial
  TolD - Gr1 Fwd primer
tgatgcgcccgaagaaat

SEQ ID NO: 8 - DNA, 20 bp, Artificial
  TolD - Gr1 Reverse Primer
cctgcgttgaaagtgcgaat

SEQ ID NO: 9 - DNA, 17 bp, artificial
  TolD - Gr1 Probe
cgaatttcgtcggcaaa

SEQ ID NO: 10 - DNA, 19 bp, Artificial
  XylM - Gr2b Fwd primer
gaccccaatcgcgatatcg

SEQ ID NO: 11 - DNA, 19 bp, Artificial
  XylM - Gr2b Reverse Primer
ttggccgtatcgagatgga

SEQ ID NO: 12 - DNA, 19 bp, Artificial
  XylM - Gr2b Probe
ccatatcgtcacccaccac

SEQ ID NO: 13 - DNA, 17 bp, Artificial
  BiPh - Gr1a Fwd Primer
ggcggcatgcagaagtg

SEQ ID NO: 14 - DNA, 16 bp, Artificial
  BiPh - Gr1a  Reverse Primer
tgctcggcggcaaact

SEQ ID NO: 15 - DNA, 15 bp, Artificial
  BiPh - Gr1A Probe
attccgtgcaactgg

SEQ ID NO: 16 - DNA, 19 bp, Artificial
  AlkB - P g1 Fwd Primer
gaggaacaacgcctttcgc

SEQ ID NO: 17 - DNA, 24 bp, Artificial
  AlkB - P g1 Reverse Primer
gctggaggatctcattatcgaaac

SEQ ID NO: 18 - DNA, 19 bp, Artificial
  AlkB - P g1 Probe
ccgcgggcaaagcgtttgg

SEQ ID NO: 19 - DNA, 19 bp, Artificial
  AlkB - P g2 Fwd Primer
gaggaacaacgcctttcgc

SEQ ID NO: 20 - DNA, 23 bp, Artificial
  AlkB - P g2 Reverse Primer
ttggttggaggatttcattatcg

SEQ ID NO: 21 - DNA, 23 bp, Artificial
  AlkB - P g2 Reverse Primer
ccgtggccaaagcgtttggagtt

SEQ ID NO: 22 - DNA, 21 bp, Artificial
  AlkB - R g1 Fwd Primer
tcgaacactacggattgctcc

SEQ ID NO: 23 - DNA, 14 bp, Artificial
  AlkB - R g1 Reverse Primer
cgggccgggctttg

SEQ ID NO: 24 - DNA, 24 bp, Artificial
  AlkB - R g1 Probe
cgcgaagacaagacggcagctttg

SEQ ID NO: 25 - DNA, 17 bp, Artificial
  AlkB - R g2 Forward primer
agtgggcggtacgagcg

SEQ ID NO: 26 - DNA, 21 bp, Artificial
  AlkB - R g2 Reverse Primer
atgttggtgcagatgtgatcg

SEQ ID NO: 27 - DNA, 21 bp, Artificial
  AlkB - R g2 Probe
ccgcaccggagcacagttgga

SEQ ID NO: 28 - DNA, 20 bp, Artificial
  ButM - AY093933 Fwd Primer
cggcgagtgtcacctcttct

SEQ ID NO: 29 - DNA, 20 bp, Artificial
  ButM - AY093933 Reverse Primer
tcccggagttccttctcgta

SEQ ID NO: 30 - DNA, 15 bp, Artificial
  ButM - AY093933 Probe
tgcaggacaccgcaa

SEQ ID NO: 31 - DNA, 30 bp, Artificial
  Methane monooxygenase (mmoX1-mmoX2) Fwd primer
cggtccgctgtggaagggcatgaagcgcgt

SEQ ID NO: 32 - DNA, 30 bp, Artificial
  mmoX1-mmoX2 Reverse Primer
ggctcgaccttgaacttggagccatactcg

SEQ ID NO: 33 - DNA, 18 bp, Artificial
  EuBac Fwd primer
cggtgaatacgttcycgg

SEQ ID NO: 34 - DNA, 19 bp, Artificial
  EuBac Reverse Primer
ggwtaccttgttacgactt

SEQ ID NO: 35 - DNA, 19 bp, Artificial
  EuBac Probe
cttgtacacaccgcccgtc

SEQ ID NO: 36 - DNA, 18 bp, Artificial
  Modified EuBac Fwd Primer
cggtgaatacgttcycgg

SEQ ID NO: 37 - DNA, 19 bp, Artificial
  Modified EuBac Reverse primer
ggwtaccttgttacgactt

SEQ ID NO: 38 - DNA, 19 bp, Artificial
  Modified EuBac Probe
cttgtacacaccgcccgtc

SEQ ID NO: 39 - DNA, 18 bp, Artificial
  Total bacteria small-subunit rRNA -Fwd Primer
cggtgaatacgttcycgg

SEQ ID NO: 40 - DNA, 20 bp, artificial
  Total bacteria small-subunit rRNA - Reverse Primer
aaggaggtgatccrgccgca

SEQ ID NO: 41 - DNA, 19 bp, Artificial
  Total bacteria small-subunit rRNA - Probe
cttgtacacaccgcccgtc

SEQ ID NO: 42 - DNA, 16 bp, Artificial
  Total bacteria 16S RNA - Fwd Primer
atggytgtcgtcagct

SEQ ID NO: 43 - DNA, 15 bp, Artificial
  Total bacteria 16S RNA - Reverse Primer
acgggcggtgtgtac

SEQ ID NO: 44 - DNA, 15 bp, Artificial
  Total bacteria 16S RNA - Probe
caacgagcgcaaccc

(19) United States
(12) Patent Application Publication
(10) Pub. No.: US 2010/0279290 A1
(43) Pub. Date: Nov. 4, 2010

(54) MICROBIOLOGICAL DETECTION METHOD

(75) Inventors: Robert Sleat, Cardiff (GB); Richard Hatton, Conwy (GB)

Correspondence Address: ROBERTS MLOTKOWSKI SAFRAN & COLE, P.C.;Intellectual Property Department, P.O. Box 10064, MCLEAN, VA, 22102-8064, US

(73) Assignee: ENVIROGENE LTD., Hengoed (GB)

(21) Appl. No.: 12/670,591

(22) PCT Filed: Jul. 28, 2008
(86) PCT No.: PCT/GB2008/002576

§ 371 Date: Jun. 8, 2010

(30) Foreign Application Priority Data
Jul. 26, 2007 (GB) 0714601.2

(51) Int. Cl. C12Q 1/68

(52) U.S. Cl. 435/6

(57) ABSTRACT

A method for detecting the presence of a hydrocarbon deposit (2) in a geographical location (1). The method comprises the steps of: detecting the presence, at the location, of a target polynucleotide encoding a protein capable of metabolising a hydrocarbon, wherein the presence of the target polynucleotide is indicative of the presence of the hydrocarbon deposit; determining the concentration of the target polynucleotide at the location; and determining a value related to the concentration of bacteria at the location and calculating the ratio of the concentration of the target polynucleotide to the value.

The present invention relates to a method for detecting the presence of a hydrocarbon deposit and, more specifically, a method of detecting a naturally occurring hydrocarbon deposit in a geographical location.

The increasing demand for fossil fuels gives rise to an ongoing need to find new, naturally occurring deposits of oil and gas which can be industrially extracted. There are many known methods of prospecting for oil and gas such as by analysing the geology of a location suspected of containing a hydrocarbon deposit. For example, a gravity or magnetic survey may be used or, for a particularly promising location, a seismic survey may be carried out.

It is also known that significant hydrocarbon deposits can result in visible surface features such as oil and natural gas seeps. However, even if such seeps are not visible, it is possible to prospect for hydrocarbon deposits by detecting the seeps in other ways. For example, WO-A-91/02086 reports on the detection of oil and gas deposits by taking soil samples from a location potentially containing a hydrocarbon deposit. This approach is based on the theory that microbes, in particular bacteria, which are capable of metabolising hydrocarbons have a selective advantage in areas where subsurface hydrocarbon gases are present. Therefore the concentration of such microbes is higher than average in a soil sample which is located above an oil or gas deposit.

In this approach, a first portion of a soil sample obtained from a location is exposed to a hydrocarbon gas such as ethane and the amount of a metabolite resulting from the metabolism of the hydrocarbon gas is measured over a predetermined length of time. This gives an indication of the activity of microbes within the soil sample which are capable of metabolising the hydrocarbon gas. A second portion of the soil sample is exposed to a substrate, such as glucose, which can generally be metabolised by all bacteria and the production of the metabolite thereof is also measured. This gives an indication of the overall microbial population in the soil sample. The ratio of the microbial population which is capable of metabolising hydrocarbons to the overall microbial population is calculated to provide a normalised index of the presence of microbes which are capable of metabolising hydrocarbons. The detection of a high index is indicative of the presence of a hydrocarbon deposit at the location. Furthermore, a plurality of samples can be taken at different sites across a location and the index determined at each site. The variation of the index across the location can then be mapped out to indicate the presence of a deposit within the location.

WO-A-91/02086 also reports on combining the index with the concentration of free hydrocarbon gas at each site in order to define more clearly areas of intense and continuous hydrocarbon flux to the surface.

The problem with the oil and gas exploration approach reported in WO-A-91/02086 is that, in practice, it is beset with a number of technical difficulties. For example, in many cases, a location which holds a potential hydrocarbon deposit is very remote and therefore it is usually some time between a soil sample being obtained from a location and the sample being analysed under laboratory conditions. During this period of time, the ratio of the number of hydrocarbon metabolising microbes to the overall number of microbes will tend to fall as the microbes are withdrawn from their source of hydrocarbons. Thus this approach can tend to give false results. Another problem is that the detection process requires the provision of radioactive hydrocarbon isotopes such as carbon 14 so that the (isotopic) metabolic products can be detected and distinguished from the products of other metabolic processes. However, radioactive isotopes require careful storage, handling and disposal. Furthermore, supplies of hydrocarbon isotopes can easily become contaminated with compounds that disrupt the normal metabolism of microorganisms leading to inaccurate results.

US2002/0065609A1 reports a different approach to mineral exploration. It discloses the analysis of microbial populations in relation to sequences of their small subunit ribosomal DNA (rDNA) sequence. It hypothesises that specific polymorphisms of the 16S rDNA sequence of bacteria can be correlated to a sample parameter such as the geographical location of populations of bacteria. If the sample parameter is the presence of hydrocarbon deposits at geographical locations then the presence of bacteria containing the polymorphisms at a geographical location is indicative of the presence of a hydrocarbon deposit at the geographical location. That is to say, specific 16S rDNA polymorphisms are putative markers for bacteria suited for survival around hydrocarbon deposits.

However, there is a problem with the approach reported in US2002/0065609A1. The 16S rDNA gene simply encodes the 16S subunit rRNA. Although the gene is highly conserved between taxonomic groups, it is not related, per se, to the ability of a microbe to survive and reproduce in an environment with a higher than average concentration of free hydrocarbon gas. Therefore, since there is no direct link between the supposed marker and the environmental survival trait, this approach is not expected to be very reliable.

WO2005/103284 relates to multi-targeted microbial screening and monitoring methods. It involves testing for the presence/absence of microbial markers that are shared by both ‘target’ and ‘index’ microbes. Index microbes are genetically distinct from target microbes but behave in a similar way under equivalent conditions. The results are used to calculate an aggregate index value. The index values are useful when the number of markers detected is not sufficient to indicate the presence of the target microbe. A threshold index value can be calculated, and if the index value is above the threshold is it indicative of the presence of target microbes.

Another approach for the detection of hydrocarbons is provided in WO03/012390. It discloses the use of micro-arrays to analyse samples for the presence of hydrocarbons and to perform multiple tests in parallel. The probes used specifically bind to analytes of targets associated with hydrocarbons.

However, the results obtained via the methods of WO2005/103284 and WO03/012390 do not distinguish between the possible causes for an increase in the number of target/hydrocarbon metabolising genes detected in the bacterial population, i.e. they do not indicate whether the detected increase is due to changes in conditions which are favourable to all bacteria in general, or whether it is caused by changes in conditions that are favourable to hydrocarbon metabolising bacteria only.

The present invention seeks to alleviate one or more of the above problems.

According to one aspect of the present invention, there is provided a method for detecting the presence of a hydrocarbon deposit in a geographical location comprising: detecting the presence, at the location, of a target polynucleotide encoding a protein capable of metabolising a hydrocarbon, wherein the presence of the target polynucleotide is indicative of the presence of the hydrocarbon deposit.

The method includes analysis of a previously obtained sample (e.g. transported to a different location) as well as analysis in situ.

According to another aspect of the present invention, there is provided a method for detecting the presence of a hydrocarbon deposit in a geographical location comprising: detecting, in a soil sample obtained from the location, the presence of a target polynucleotide encoding a protein capable of metabolising a hydrocarbon in the sample, wherein the presence of the target polynucleotide is indicative of the presence of the hydrocarbon deposit.

According to another aspect of the present invention, there is provided a method for detecting the presence of a hydrocarbon deposit in a geographical location comprising the steps of:

detecting the presence, at the location, of a target polynucleotide encoding a protein capable of metabolising a hydrocarbon;
determining the concentration of the target polynucleotide at the location; and
determining a value related to the concentration of microbes such as bacteria at the location and calculating the ratio of the concentration of the target polynucleotide to the value, the ratio being indicative of the presence or absence of a hydrocarbon deposit.

In some embodiments, the method involves the detection of only a fragment of the target polynucleotide, the fragment identifying the target polynucleotide. That is to say the invention relates to detecting the presence of at least a fragment of the target polynucleotide. The fragment may be at least 10, 15, 20, 30 or 50 nucleotides long.

Preferably, the hydrocarbon deposit is a naturally occurring hydrocarbon deposit.

Conveniently, the method further comprises the step of determining the concentration of the target polynucleotide at the location.

Preferably, the method further comprises the step of determining a value related to the concentration of microbes such as bacteria at the location and calculating the ratio of the concentration of the target polynucleotide to the value.

Advantageously, the step of determining the value related to the concentration of bacteria at the location comprises the step of determining the concentration of a generic polynucleotide present in a plurality of different types of bacteria.

Advantageously, the presence of the generic polynucleotide, such as small subunit rRNA, is determined using a forward primer comprising one of SEQ ID NOs: 33 or SEQ ID NO: 39, or a sequence with at least 80% identity thereto, a reverse primer comprising SEQ ID NO:34 or SEQ ID NO: 40 respectively, or a sequence with at least 80% identity thereto, and optionally a probe comprising SEQ ID NO:35 or SEQ ID NO: 41 respectively, or a sequence with at least 80% identity thereto.

Conveniently, the presence of the generic polynucleotide, such as 16s rRNA, is determined using a forward primer comprising SEQ ID NO:36 or SEQ ID NO: 42, or a sequence with at least 80% identity thereto, a reverse primer comprising SEQ ID NO:37 or SEQ ID NO:43 respectively, or a sequence with at least 80% identity thereto, and optionally a probe comprising SEQ ID NO:38 or SEQ ID No:44 respectively, or a sequence with at least 80% identity thereto.

Conveniently, the step of detecting the presence of the target polynucleotide comprises detecting the presence of a subsequence of the target polynucleotide sequence.

Preferably, the subsequence comprises a consensus sequence present in a plurality of different genes encoding a protein capable of metabolising a hydrocarbon.

Advantageously, the target polynucleotide is DNA.

Alternatively, the target polynucleotide is RNA.

Conveniently, the hydrocarbon is a C1 to C20 alkane, an alkene, an optionally substituted single or multi-ring aromatic hydrocarbon, or a naphthene, preferably a C2 to C20 alkane.

Preferably, the protein capable of metabolising a hydrocarbon is a biphenyl dioxygenase, a toluene monooxygenase, an alkane hydroxylase; a catechol 2,3,dioxygenase; a naphthalene dioxygenase; a toluene dioxygenase; a xylene monooxygenase; a butane monooxygenase; a bacterial P450 oxygenase; a eukaryotic P450 oxygenase; or a alkane dehydrogenase.

Conveniently, the protein capable of metabolising a hydrocarbon is enclosed by a nucleotide sequence comprising a sequence with at least 80% sequence identity to a sequence referred to in Table 2. It is preferred that the sequence has at least 90%, 95%, 99% or 100% sequence identity to a sequence referred to in Table 2. Table 2 provides the GenBank accession numbers of the sequences. The preferred sequences are those present in the GenBank Database on 28 Jul. 2008.

Advantageously, the presence of the biphenyl dioxygenase protein is determined using a forward primer comprising SEQ ID NO:13 or a sequence with at least 80% identity thereto, a reverse primer comprising SEQ ID NO:14 or a sequence with at least 80% identity thereto, and optionally a probe comprising SEQ ID NO:15 or a sequence with at least 80% identity thereto.

Conveniently, the presence of the catechol 2,3,dioxygenase protein is determined using a forward primer comprising SEQ ID NO:1 or a sequence with at least 80% identity thereto, a reverse primer comprising SEQ ID NO:2 or a sequence with at least 80% identity thereto, and optionally a probe comprising SEQ ID NO:3 or a sequence with at least 80% identity thereto.

Preferably, the presence of the naphthalene dioxygenase protein is determined using a forward primer comprising SEQ ID NO:4 or a sequence with at least 80% identity thereto, a reverse primer comprising SEQ ID NO:5 or a sequence with at least 80% identity thereto, and optionally a probe comprising SEQ ID NO:6 or a sequence with at least 80% identity thereto.

Advantageously, the presence of the toluene dioxygenase protein is determined using a forward primer comprising SEQ ID NO:7 or a sequence with at least 80% identity thereto, a reverse primer comprising SEQ ID NO:8 or a sequence with at least 80% identity thereto, and optionally a probe comprising SEQ ID NO:9 or a sequence with at least 80% identity thereto.

Conveniently, the presence of the xylene monooxygenase protein is determined using a forward primer comprising SEQ ID NO:10 or a sequence with at least 80% identity thereto, a reverse primer comprising SEQ ID NO:11 or a sequence with at least 80% identity thereto, and optionally a probe comprising SEQ ID NO:12 or a sequence with at least 80% identity thereto.

Preferably, the presence of the butane monooxygenase protein is determined using a forward primer comprising SEQ ID NO:28 or a sequence with at least 80% identity thereto, a reverse primer comprising SEQ ID NO:29 or a sequence with at least 80% identity thereto, and optionally a probe comprising SEQ ID NO:30 or a sequence with at least 80% identity thereto.

Advantageously, the presence of the alkane dehydrogenase protein is determined using a forward primer comprising one of SEQ ID NOs: 16, 19, 22 or 25, or a sequence with at least 80% identity thereto, a reverse primer comprising SEQ ID NO:17, 20, 23 or 26 respectively, or a sequence with at least 80% identity thereto, and optionally a probe comprising SEQ ID NO:18, 21, 24 or 27 respectively, or a sequence with at least 80% identity thereto.

Conveniently, the presence of the methane monooxygenase protein, is determined using a forward primer comprising SEQ ID NO:31, or a sequence with at least 80% identity thereto, and a reverse primer comprising SEQ ID NO:32 respectively, or a sequence with at least 80% identity thereto.

Alternatively, the forward primers, reverse primers and probes used for the detection of the hydrocarbon metabolising and generic polynucleotide genes have 80, 85, 90, 95, 99 or 99.5% identity with the respective SEQ ID NOs.

In this specification, the percentage “identity” between two sequences is determined using the BLASTP algorithm version 2.2.2 (Altschul, Stephen F., Thomas L. Madden, Alejandro A. Schäffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402) using default parameters. In particular, the BLAST algorithm can be accessed on the internet using the URL http://www.ncbi.nlm.nih.gov/blast/.

Advantageously, the target polynucleotide is an alkB gene.

Conveniently, the presence of the target polynucleotide is determined using PCR.

Preferably, the concentration of the target polynucleotide and optionally the concentration of the generic polynucleotide is determined by quantitative PCR.

Advantageously, the step of detecting the presence of the target polynucleotide comprises obtaining a soil sample from the location and detecting the presence of the target polynucleotide in the soil sample.

Conveniently, the soil sample is obtained from a depth of between 10 and 50 cm below the surface.

Preferably, the method further comprises the step of, after obtaining the soil sample, stabilising the nucleic acids in the soil sample.

Advantageously, the method comprises the step of obtaining a plurality of soil samples at different sites at the geographical location and carrying out the method on each soil sample.

Conveniently, the method further comprises the step of correlating the results of the method on each soil sample, thereby determining the variation in the presence of the target polynucleotide at different sites within the geographical location.

Preferably, the method further comprises the steps of determining the concentration of the target polynucleotide and generating a first index related to said concentration at each site; determining the concentration of free hydrocarbon gas above each site and generating a second index related to said free hydrocarbon gas concentration at each site; and combining the first and second indexes for each site at the geographical location.

In this specification the term “hydrocarbon” means an organic chemical compound comprising hydrogen and carbon atoms.

In this specification, where a protein is described as being “capable of metabolizing a hydrocarbon” this means that the protein has activity in facilitating or causing a chemical reaction on a hydrocarbon under in vivo conditions. This can be tested, for example, by adding a sample hydrocarbon to a reaction medium that replicates intracellular conditions and detecting the decrease in the presence of the hydrocarbon over a predetermined length of time in comparison with a control medium from which the protein is absent.

Embodiments of the present invention will now be described with reference to the accompanying figures in which:—

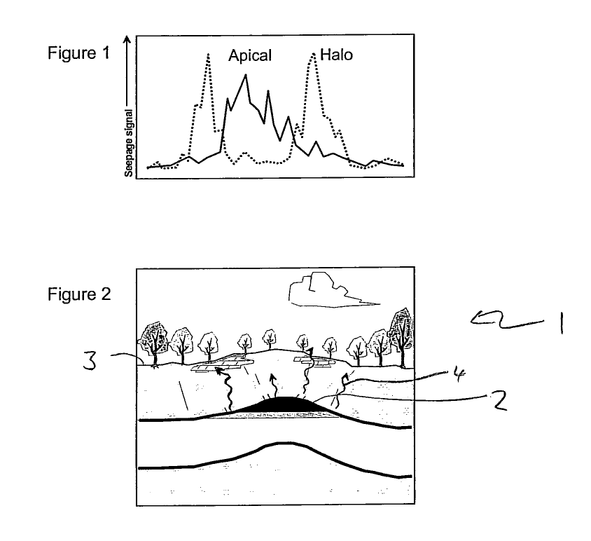

FIG. 1 is a graph showing free gas concentrations in surface soils versus location;

FIG. 2 is a schematic cross-sectional view of a geographical location containing a subsurface hydrocarbon deposit;

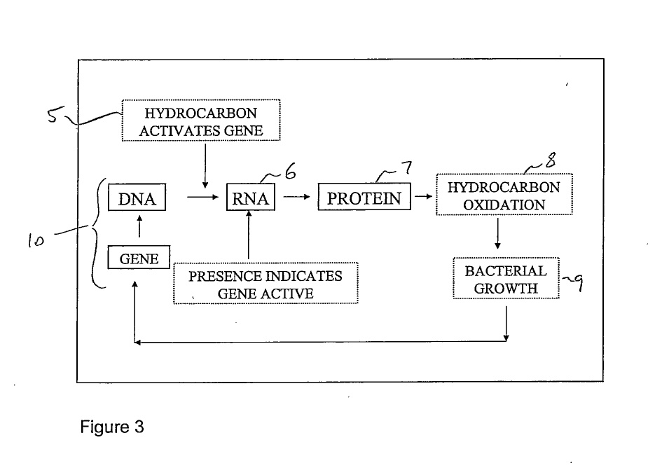

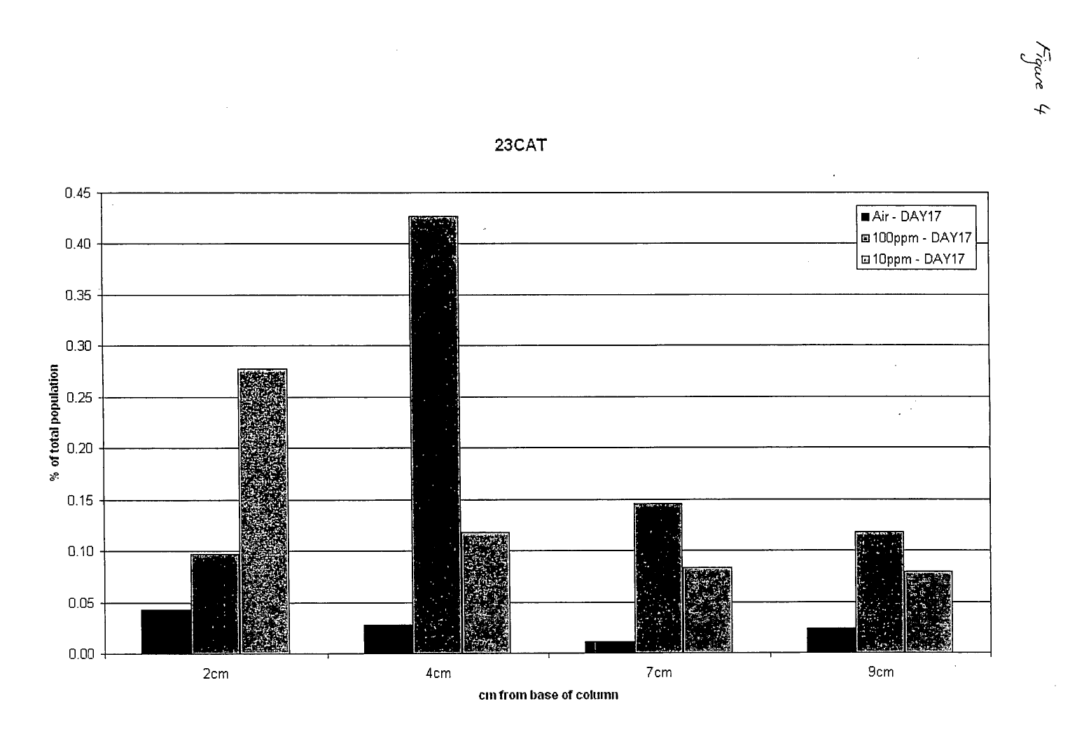

FIG. 3 is a block diagram demonstrating the principle of the present invention;

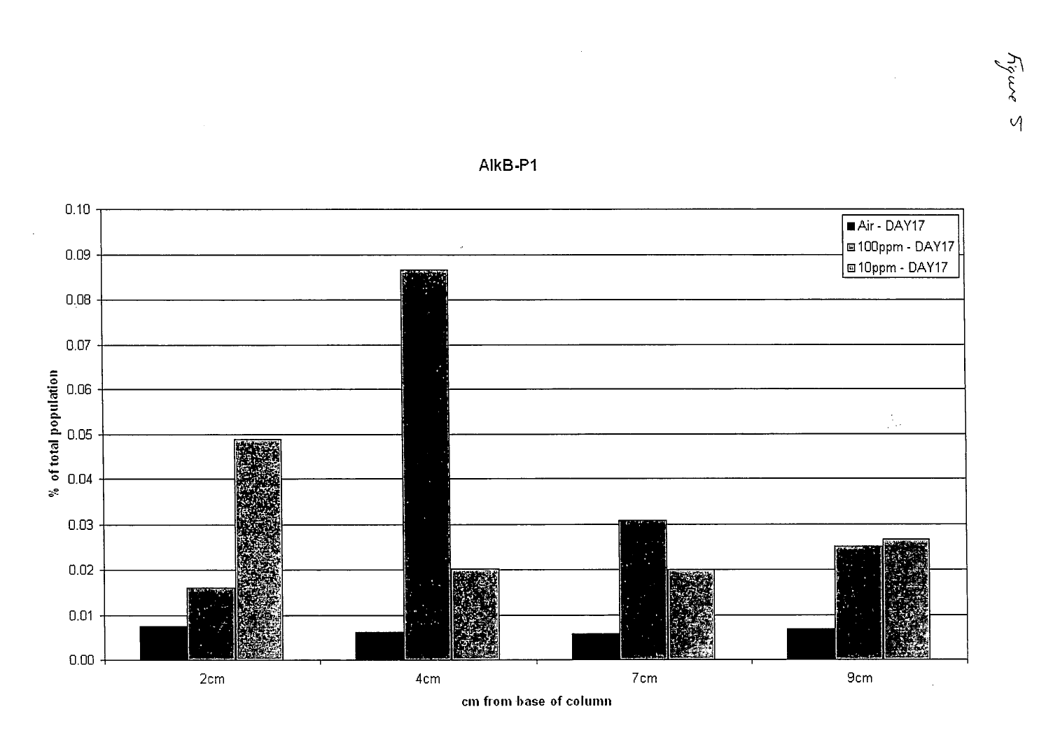

FIG. 4 is a graph showing normalized data obtained from a 2,3, CAT assay of soil samples;

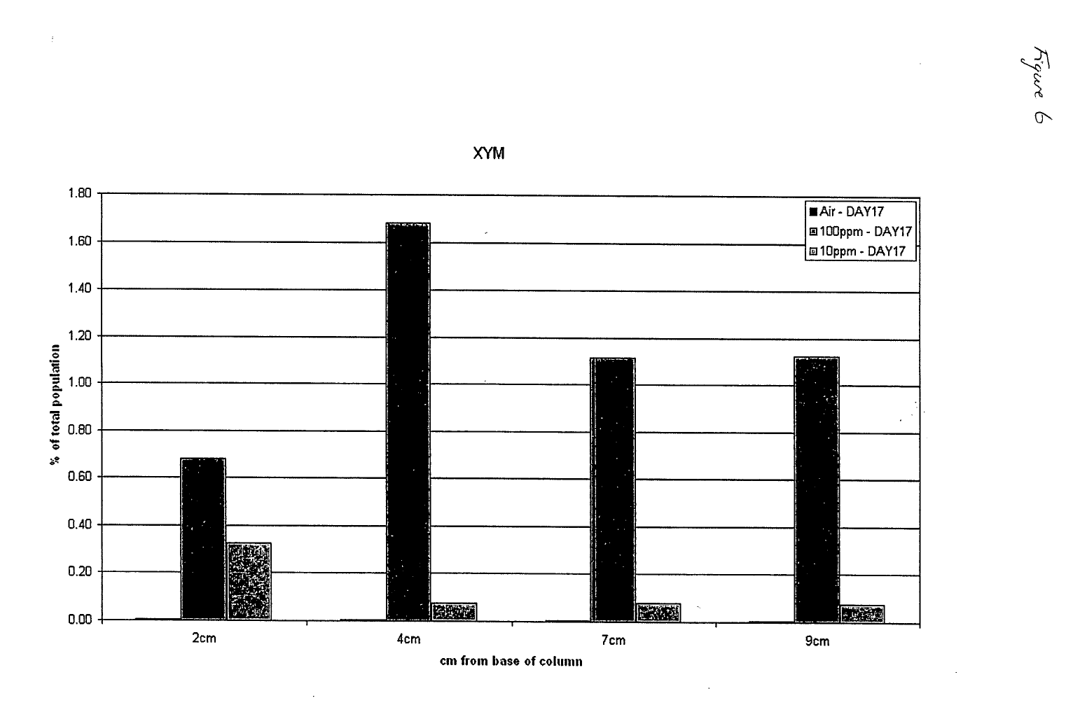

FIG. 5 is a graph showing normalized data obtained from an AlkB-P1 assay of soil samples; and

FIG. 6 is a graph showing normalized data obtained from an XyM assay of soil samples.

Referring to FIGS. 1 and 2, the principle that underlies the present invention will be described.

At a geographical location 1, a subsurface hydrocarbon deposit 2 is not visible from the surface 3. However, the vertical migration of hydrocarbon gases 4 to the surface 3 from the hydrocarbon deposit results in the generation of an anomaly at the surface 3 of increased concentrations of hydrocarbon gas in surface soils. In some situations, the anomaly is directly above the hydrocarbon accumulation (an apical anomaly) but in other situations, the anomaly takes the form of a halo around the periphery of the hydrocarbon deposit (a halo anomaly). Referring to FIG. 1, the concentration of hydrocarbon gas across the geographical location 1 either as an apical or a halo anomaly is shown.

The presence of hydrocarbon gas in surface soils results in the development of soil microbial populations capable of using the hydrocarbons as a nutrient and energy source. Thus the presence of these bacterial populations in soils (or, to be precise, the presence of elevated concentrations of these bacterial populations) indicates that hydrocarbon gases are migrating through the soils. This, in turn, indicates that there is an underlying oil or gas reservoir.

Therefore, the present invention concerns detecting the presence of microbial and, in particular, bacterial populations which are capable of metabolising hydrocarbons.

### Detection of Microbial Populations

In embodiments of the present invention, microbial populations in the vicinity of a hydrocarbon deposit are detected by detecting the presence of genes which encode a protein capable of metabolising a hydrocarbon. The principle which lies behind this concept will now be described with reference to FIG. 3 which shows a block diagram indicating the relationship between the presence of hydrocarbons and the expression of genes encoding hydrocarbon metabolising proteins.

Starting at block 5, the presence of a hydrocarbon in the environment surrounding a microbe (in particular a bacterium) is detected by the microbial cell via a number of mechanisms, principally via receptors on the cell surface. The detection of the presence of hydrocarbon results in a raised level of a transcription of mRNA 6 which encodes a protein capable of metabolising the hydrocarbon. The protein 7, is in turn, translated and expressed within the cell. The protein duly oxidises the hydrocarbon 8 releasing energy and resulting in growth 9 and multiplication of the cell. The multiplication of the cell, leads to an increase in the number of genes 10 encoding hydrocarbon metabolising proteins in that locale, i.e. an increase in the concentration of such genes.

In contrast, a microbe which does not contain a gene encoding a hydrocarbon metabolising protein does not metabolise hydrocarbons and does not benefit from the hydrocarbons as a source of energy in the environment. Thus where hydrocarbon deposits are present, the overall microbial population is skewed in favour of microbes containing genes encoding hydrocarbon metabolising proteins. Therefore, the concentration of DNA in a sample population is skewed in favour of DNA encoding hydrocarbon metabolising proteins.

In specific embodiments of the present invention, the concentration of genes encoding hydrocarbon metabolising proteins varies not only due to the number of cells containing such genes in the population but also the number of copies of such genes within each cell. For example bacterial cells containing a plasmid with such a gene will have a selective advantage in a hydrocarbon-rich environment over cells which do not contain such a plasmid. Furthermore, a cell which contains multiple plasmids incorporating such a gene may have a selective advantage over a cell containing plasmid with only a single copy of a hydrocarbon metabolising protein encoding gene. Therefore, in such embodiments, the detection of genes encoding hydrocarbon metabolising proteins is particularly sensitive.

The above described embodiments relate to the detection of DNA encoding hydrocarbon metabolising proteins. However, in some alternative embodiments, RNA and, in particular, mRNA molecules encoding hydrocarbon metabolising proteins are detected instead. It is to be appreciated that mRNA transcripts exist for a relatively short period of time within a cell and therefore the detection of such an mRNA transcript is indicative of the cell actively metabolising hydrocarbons at the time of sampling. In contrast, the DNA gene copy number is an integrative measure of the embodiment's exposure to specific hydrocarbons over a period of time.

In further embodiments of the present invention, the relative concentrations of DNA and mRNA encoding hydrocarbon metabolising proteins are compared to give an indication of the history of the presence of hydrocarbons in the environment of the microbial population. For example, if the concentration of DNA encoding hydrocarbon metabolising proteins in a microbial population is found to be average but the concentration of mRNA encoding hydrocarbon metabolising proteins is found to be well above average then this may be an indication that there is no underlying hydrocarbon deposit in the environment of the microbial population and that the presence of the high concentration of mRNA transcripts is due to human intervention (e.g. the vehicle of an individual taking the samples).

### Marker Genes

In order to carry out embodiments of the present invention, it is necessary to identify hydrocarbons which are metabolised by microbes; proteins which effect the oxidation process; and genes which encode the proteins.

The number of organic compounds found in crude oil numbers in the thousands. While there are far fewer compounds found in thermogenically derived gas, they still number in the hundreds. Hydrocarbons which migrate from a naturally occurring subsurface reservoir are grouped into two categories based, not on their structure, but on their volatility, namely volatiles and semi-volatiles. The volatiles are generally found as gases at standard temperature and pressure conditions while semi-volatiles are generally liquid under similar conditions but can volatilise very easily.

The main chemical component of gas migrating from a naturally occurring sub-surface reservoir to the surface is methane. Methane can be generated geogenically and biogenically, and therefore there is a risk of “false positive” results if detecting the presence of methane. However, it is still a useful marker gene of the present invention. In preferred embodiments, a C2 to C20 alkane is detected (longer chained alkanes are found at decreasing concentrations of migrating gas). In other embodiments of the invention, straight chain alkanes and branch chain alkanes are detected instead. In further embodiments, alkenes are detected or, alternatively, simple and alkylated single multi-ring aromatics and saturated rings (napthenes) are detected.

### Alkane Oxidation

The enzymology of microbial alkane oxidation is well known in the art with many reviews available (see for example Oil & Gas Science and Technology—Rev. IFP, Vol. 58 (2003) pp 427-440).

Straight-chain hydrocarbons are oxidized by a group of enzymes known as alkane hydroxylases. These enzymes introduce oxygen atoms derived from molecular oxygen into the alkane substrate. Alkane degrading yeast strains contain multiple alkane hydroxylases belonging to the P450 superfamily, while many bacteria contain membrane-bound alkane hydroxylase systems. Short-chain alkanes are thought to be oxidized by alkane hydroxylases related to the soluble and particulate methane monooxygenases.

Some embodiments involve the detection of methane monooxygenases, e.g. the detection of soluble methane monooxygenase (sMMO) using primers mmoX1-mmoX2 (see Table 1), as described in Miguez et al., Microbiol Ecology (1997), 33:21-31. Embodiments comprising assays based on the membrane bound alkane hydroxylases (alkB) are preferred as the enzyme is thought to target longer chain alkanes.

### Aromatic Oxidation

The enzymology of microbially mediated aromatic oxidation is also well known in the art

Most aerobic aromatic-hydrocarbon biodegradation pathways converge through catechol-like intermediates that are typically cleaved by ortho- or meta-cleavage dioxygenases. Catechol 2,3 dioxygenases (C23DO) may thus represent a generic enzyme group for the metabolism of aromatics.

The individual pathways of aromatic biodegradation are usually initiated through the action of either a dioxygenase or a monooxygenase. For example biphenyl dioxygenase is involved in the oxidative biodegradation of phenol whilst toluene monooxygenase is involved in the oxidative biodegradation of toluene.

References of reports on detection of genes responsible for aromatic oxidation in environmental samples include the following: Applied & Environmental Microbiology, 65, 80-87 (1999); Applied & Environmental Microbiology, 56, 254-259 (1990); Applied & Environmental Microbiology, 67, 1542-1550 (2001); Applied & Environmental Microbiology, 66, 80-8678-6837 (2000); and Applied & Environmental Microbiology, 69, 3350-3358 (2003)

### Naphthenes

Naphthenes are saturated single or multi-ring aromatics, which can be modified with alkyl substituents

Therefore, preferred embodiments of the present invention involve the detection of genes encoding one of the following enzymes: Alkane hydroxylase (alkB related); Catechol 2,3 dioxygenase; Napthalene dioxygenase; Toluene monooxygenase; Toluene dioxygenase; Xylene monooxygenase, or biphenyl dioxygenase.

Other suitable genes are those which encode one of the following enzymes: Butane monooxygenases (similar to pMMO and sMMO); Bacterial P450 oxygenases (C4-C16 n-alkanes); or Eukaryotic P450 oxygenases (C10-C16 n-alkanes).

### Identifying Genes

Further details of such exemplary enzymes and the primers that can be used for their identification are provided in Table 1. However, it is to be appreciated that the genes referred to in Table 1 are by no means exhaustive and further genes could be used instead. Other suitable marker genes are identified by, for example, searching public databases (e.g. GenBank and Ribosomal Database Project) for genes reported to encode hydrocarbon-metabolising proteins. Having identified a plurality of genes in this way, it is preferred that the genes are aligned and areas of homology located in order to identify potential motifs that characterize genes encoding proteins that have this functionality. Such potential motifs are then compared with gene databases and those potential motifs that are found in genes encoding proteins not associated with hydrocarbon metabolism are discarded. Confirmed motifs (that is to say, motifs only found in genes encoding hydrocarbon-metabolising proteins) are then used as target polynucleotides in the method of the present invention.

For example, the DNA sequence(s) coding for a specific catabolic gene can be searched for in the GenBank (http://www.ncbi.nlm.nih.gov/), e.g. see Table 2, and imported into software for manipulating DNA sequences (such as DS Gene, www.accelrys.com). Using the software tools provided within the program, the sequences are aligned and phylogenetic analysis performed. These selected downloaded sequences are examined and consensus regions identified. These consensus sequences must show a high percentage of conformity for the sequences obtained for the resulting assay to be specific for the desired gene. The consensus sequence is then exported into Primer Express software (www.appliedbiosystems.com), which analyses the consensus sequence and provides suggestions of primer/probe combinations that can be used in qPCR assays.

TABLE 1

| | Assay | Forward Primer | Reverse Primer | |
|---|---|---|---|---|
| Target | Abrev | (from 5′) | (from 5′) | Probe (from 5') |
| Catechol 2-3 | 23CAT-Gr1 | GCCGCCTCCATCATGTGT | GGCGCGAAGCACGTCTT | FAM-CTTCTACCTCGAAACCT-MGB |
| dioxygenase | | (SEQ ID NO: 1) | (SEQ ID NO: 2) | (SEQ ID NO: 3) |
| Naphthalene | NapD-Gr1a | TGGGTGACGCTGCTTGGT | TCTAAACCGCCGGAATGC | FAM-CCTGGAACCTATGTTCA-MGB |
| dioxygenase | | (SEQ ID NO: 4) | (SEQ ID NO: 5) | (SEQ ID NO: 6) |
| Toluene | TolD-Gr1 | TGATGCGCCCGAAGAAAT | CCTGCGTTGAAAGTGCGAAT | FAM-CGAATTTCGTCGGCAAA-MGB |
| dioxygenase | | (SEQ ID NO: 7) | (SEQ ID NO: 8) | (SEQ ID NO: 9) |
| Xylene | XylM-Gr2b | GACCCCAATCGCGATATCG | TTGGCCGTATCGAGATGGA | FAM-CCATATCGTCACCCACCAC-MGB |
| monooxygenase | | (SEQ ID NO: 10) | (SEQ ID NO: 11) | (SEQ ID NO: 12) |
| Biphenyl | BiPh-Gr1a | GGCGGCATGCAGAAGTG | TGCTCGGCGGCAAACT | FAM-ATTCCGTGCAACTGG-MGB |
| dioxygenase | | (SEQ ID NO: 13) | (SEQ ID NO: 14) | (SEQ ID NO: 15) |
| Alkane | AlkB-Pg1 | GAGGAACAACGCCTTTCGC | GCTGGAGGATCTCATTATC | FAM-CCGCGGGCAAAGCGTTTGG- |
| dehydrogenase | | (SEQ ID NO: 16) | GAAAC | TAMRA |
| | | | (SEQ ID NO: 17) | (SEQ ID NO: 18) |
| Alkane | AlkB-Pg2 | GAGGAACAACGCCTTTCGC | TTGGTTGGAGGATTTCATT | FAM-CCGTGGCCAAAGCGTTTGGAGTT- |
| dehydrogenase | | (SEQ ID NO: 19) | ATCG | TAMRA |
| | | | (SEQ ID NO: 20) | (SEQ ID NO: 21) |
| Alkane | AlkB-Rg1 | TCGAACACTACGGATTGCTCC | CGGGCCGGGCTTTG | FAM-CGCGAAGACAAGACGGCAGCTTTG- |
| dehydrogenase | | (SEQ ID NO: 22) | (SEQ ID NO: 23) | TAMRA |
| | | | | (SEQ ID NO: 24) |
| Alkane | AlkB-Rg2 | AGTGGGCGGTACGAGCG | ATGTTGGTGCAGATGTGATCG | FAM-CCGCACCGGAGCACAGTTGGA- |
| dehydrogenase | | (SEQ ID NO: 25) | (SEQ ID NO: 26) | TAMRA |
| | | | | (SEQ ID NO: 27) |
| Butane | ButM- | CGGCGAGTGTCACCTCTTCT | TCCCGGAGTTCCTTCTCGTA | FAM-TGCAGGACACCGCAA-MGB |
| Monooxygenase | AY093933 | (SEQ ID NO: 28) | (SEQ ID NO: 29) | (SEQ ID NO: 30) |
| Methane | mmoX1- | CGGTCCGCTGTGGAAGGGCA | GGCTCGACCTTGAACTTGG | |
| monooxygenase | mmoX2 | TGAAGCGCGT | AGCCATACTCG | |
| | | (SEQ ID NO: 31) | (SEQ ID NO: 32) | |

TABLE 2
GenBank Accession Numbers of partial or complete nucleotide
sequences encoding hydrocarbon metabolising enzymes, each
of which is incorporated herein by reference.

| Target | Accession Number |
|---|---|
| Catechol 2-3 dioxygenase | AJ544931 |
| | AJ544928 |
| | AJ544927 |
| | AJ544926 |
| | AJ544936 |
| | AJ544935 |
| | AJ544937 |
| | AJ544933 |
| | AJ544929 |
| Naphthalene dioxygenase | AY694167 |
| | AY694169 |
| | AY694170 |
| | AY694172 |
| | AY694166 |
| | AY694168 |
| | AY694165 |
| | AY694171 |
| | AY694164 |
| Toluene Dioxygenase | AJ512673 |
| | AJ512671 |
| | AJ512672 |
| Xylene Monooxygenase | DD317812 |
| | DD180886 |
| Biphenyl Dioxygenase | DQ521945 |
| | DQ521940 |
| | DQ521941 |
| | DQ521942 |
| | DQ521939 |
| | DQ521946 |
| Butane monooxygenase | AY093933 |
| Alkane Dehydrongenase - P1 | AJ233397 |
| | AJ833927 |
| | AJ250560 |
| | AY034587 |
| Alkane Dehydrongenase - P2 | AY286497 |
| | AJ245436 |
| | AJ344083 |
| Alkane Dehydrongenase - R1 | AJ833979 |
| | AJ301875 |
| | AY452488 |
| | AJ301874 |
| | AJ833977 |
| Alkane Dehydrongenase - R2 | AJ301867 |
| | AJ301866 |
| | AJ833985 |

### Microbial Population Size Marker Genes

Embodiments of the present invention involve identifying the concentration of a gene encoding a hydrocarbon metabolizing protein in a sample. However, it is preferred that the concentration is determined with reference to the total microbial population in the sample so as to give an indication of the relative concentration of such genes which respect to the total microbial population. There are various means that can be employed in order to determine the total microbial population such as measuring the conversion of a generic substrate such as glucose to carbon dioxide by microbes in the sample; classical plate counts; and quantification of bacterial polymers (e.g. peptidoglycan).

However, the quickest and most preferred approach is to measure the presence of generic oligonucleotide sequences which are present in all or almost all microbes, irrespective of their capacity to metabolize hydrocarbons. Examples of suitable genes from a bacterial population are provided in Table 3. Two EuBac genes are provided in Table 3. The first is that provided and used in the assay described in Suzuki et al. 2000, AEM, 66(11) p4605-4614. The second is used in a modified assay which optimises the results obtained in the normalisation assays. There is usually 10³-10⁶ more of this generic gene present in the bacteria than the indicator gene, and it is unaffected by the presence of hydrocarbons or other environmental conditions in the soil. In the modified version the same EuBac primer sequences have been used as reported in Suzuki et al., and the probe sequence is as reported with the exception that a FAM-minor groove binder probe has been used rather than the FAM-TAMRA probe. In addition, when the modified EuBac assay is run the PCR thermo-cycling conditions differ from those described in Suzuki et al. The modified cycle conditions comprise 10 minutes at 95° C. followed by 40 cycles of 95° C. for 15 seconds and 57° C. for 1 minute.

It is also to be noted that in some embodiments, the microbial population is determined by carrying out quantitative PCR using generic primers of oligonucleotide sequences that vary slightly between strains of bacteria. Because the primers are generic, amplification of the oligonucleotide sequences takes place and is indicative of the microbial population notwithstanding differences between the oligonucleotide sequences of different bacterial strains.

It is to be appreciated that other suitable generic nucleotides sequences could be used instead of those disclosed in Table 3. Such sequences can be identified by, for example searching public databases for nucleotide motifs which are present in a high proportion of microorganisms, e.g. at least 80% of microorganisms.

TABLE 3

| | Gene | Forward Primer | TM | Reverse Primer | TM | | TM | |
|---|---|---|---|---|---|---|---|---|
| Target | Name | (from 5′) | ° C. | (from 5′) | °C. | Probe (from 5′) | ° C. | Ref |
| Total | Small- | CGGTGAATAC | | GGWTACCTTGT | | FAM-CTTGTACACACCGCCC | | Suzuki et al. 2000, |
| Bacteria | subunit | GTTCYCGG | | TACGACTT | | GTC-TAMRA | | Applied & Environmental |
| (Eubac) | rRNA | (SEQ ID NO: 33) | | (SEQ ID NO: 34) | | (SEQ ID NO: 35) | | Microbiology 66(11) |
| | | | | | | | | p4605-4614 |
| Total | 16s | Bac1369- | 59.2 | Prok1492- | 44.3 | TM1389- | 73.0 | Suzuki et al. 2000, |
| Bacteria | rRNA | CGGTGAATAC | | GGWTACCTTGT | | CTTGTACACACCGCCCGTC | | Applied & Environmental |
| (EuBac) | | GTTCTCGG | | TACGACTT | | (SEQ ID NO: 38) | | Microbiology 66(11) |
| | | (SEQ ID NO: 36) | | (SEQ ID NO: 37) | | | | p4605-4614, plus |
| | | | | | | | | modifications |
| Total | Small- | CGGTGAATAC | | AAGGAGGTGAT | | FAM-CTTGTACACACCGCCC | | Suzuki et al., 2000, |
| bacteria | subunit | GTTCTCGG | | CCTGCCGCA | | GTC-TAMRA | | Applied & Environmental |
| | rRNA | (SEQ ID NO: 39) | | (SEQ ID NO: 40) | | (SEQ ID NO: 41) | | Microbiology 66(11) |
| | | | | | | | | p4605-4614 |
| Total | 16S | ATGGYTGTCG | | ACGGGCGGTGT | | FAM-CAACGAGCGCAACCC | | Ritalahti et al. 2006, |
| bacteria | RNA | TCAGCT | | GTAC | | TAMRA | | Applied & Environmental |
| | | (SEQ ID NO: 42) | | (SEQ ID NO: 43) | | (SEQ ID NO: 44) | | Microbiology 72(4) |
| | | | | | | | | p2765-2774 |

### Gene Quantification

In embodiments of the present invention, the concentration of a gene encoding a hydrocarbon metabolizing protein and, preferably, the concentration of a generic microbial gene in a sample is determined. The preferred technique for determining these concentrations is quantitative polymerase chain reaction (qPCR) which is also known as “real time PCR” (see, for example, Ding C et al “Quantitative Analysis of Nucleic Acids—The Last Few Years of Progress” J. Biochem Mol. Biol. 2004 Jan. 31; 37 (1):1-10).

The principle underlying qPCR is that during the course of a PCR assay, the number of amplicons generated is monitored PCR cycle by PCR cycle. This is usually achieved by introducing a fluorophor into the assay system. The amount of fluorescence generated is directly proportional to the number of amplicons generated at each PCR cycle whilst the number of amplicons is a function of starting copy number and the number of PCR cycles. Therefore, by measuring the intensity of the signal (e.g. fluorescence) as the PCR cycles progress, the starting concentration of a target sequence may be determined. In particular, the PCR is monitored during the exponential phase where the first significant increase in the amount of PCR product correlates to the initial amount of target template. The higher the starting copy number of the nucleic acid target, the sooner a significant increase in fluorescence is observed. A significant increase in fluorescence above the baseline value indicates the detection of accumulated PCR product (measured by the Ct value).

Absolute quantitation in PCR requires a standard curve of known copy numbers, which can be constructed using a synthesized oligonucleotide or amplicon. This amplicon is of a known concentration and by serial dilution can give a wide range of known standards. These standards then undergo PCR using exactly the same conditions as the target DNA sequence. The copy number of the target DNA sequence is then extrapolated in the sample from the calibration graph or standard curve, which is constructed plotting the log of copy number against the Ct value.

Exemplary apparatus includes the ABI 7300 sequence detector which performs 96 parallel wells of qPCR analyses, determines a standard curve and calculates the amount of target DNA in each of the sample wells. Thus the output is a direct measure of the abundance of the target sequence in the sample.

While a number of variants of the chemistry of the assay system are known in the art, a Taqman 5′ nuclease assay or a SyBr Green system are particularly preferred. The Taqman 5′ nuclease assay is more sensitive to the presence of a target sequence and is more specific thereto but it requires three closely linked conserved regions in the target gene. The SyBr Green assay system is less sensitive and specific but only requires the presence of two conserved regions.

In the embodiments in which the concentration of both a gene encoding a hydrocarbon metabolizing protein and a generic gene are determined, the concentration of both genes can be determined simultaneously in a multiplex qPCR reaction. In such an assay system, forward and reverse primers and a probe are provided for both genes but the signalling system is different for each probe (e.g. the label fluoresces at a different wavelength) so that the relative quantities of each gene can be determined independently during PCR.

The detection of generic gene sequences using Taqman qPCR assays are described in Aromatics—Applied & Environmental Microbiology, 69, 3350-3358 (2003); and 16S RNA for total microbial populations—Applied & Environmental Microbiology, 69, 6597-6604 (2003).

The detection of gene sequences using SyBr Green qPCR assays are disclosed in Environmental Microbiology, 6, 754-759 (2004); FEMS Microbiology Ecology, 41, 141-150 (2002); and Environmental Microbiology, 1, 307-317 (1999).

### Soil Sample Stabilization

It is to be appreciated that under normal conditions, the microbial population of a soil sample will change over time, once the soil sample has been removed from its original geographical location. More specifically, if a soil sample is taken from a location where a subsurface hydrocarbon deposit is present then, upon removal of the soil sample, the microbes within the soil sample are deprived of their hydrocarbon source. This may result in the relative populations of hydrocarbon and non-hydrocarbon metabolising microbes within the soil sample reverting to levels typical of a sample taken in a locality that does not have a hydrocarbon deposit. In practice, it may be necessary for samples to be taken from a locality and analysed by the methods of the present invention under laboratory conditions. It is therefore important that the gene copy numbers within the soil sample remain unchanged between removal of the sample from a location and analysis in the laboratory.

Accordingly, in some embodiments of the present invention, specific steps are taken to stabilize the presence of nucleic acids within the solid sample. In one embodiment, soil samples are kept frozen at 0° C. or, for example, in liquid nitrogen at −196° C. However, it is preferred that gene copy numbers are stabilized by the addition of a nucleic acid stabilizing compound such as RNALater™ from Ambion™ and RNAprotect™ from Qiagen™. The provision of a chemical nucleic acid stabiliser avoids the need for cumbersome freezing apparatus to be transported to the geographical location from which the soil sample is obtained. Although these stabilizing compounds are specifically designed for stabilization of RNA, which is generally less stable than DNA, the compounds are also effective in stabilizing DNA.

### Soil Sample Extraction

In embodiments of the present invention, RNA and/or DNA is extracted from soil samples. Although soil samples may contain contaminating substances which interfere with the PCR reaction and thus the quantitation process, the preferred embodiments, in which a generic microbial gene is also quantified, are not sensitive to such contaminating substances since nucleic acid concentrations are normalized with reference to the generic microbial gene sequence. Similarly, although different soil types may affect the efficiency of the extraction of nucleic acids, this can be normalized by quantification of generic microbial gene sequences in soil samples.

Kits for extracting an isolated nucleic acid from soil samples are sold commercially by MoBio Laboratories, Inc. These kits require a soil sample to be added to a bead beating tube for rapid homogenization. Cell lysis occurs by both chemical and mechanical means (vortex adapter). Total genomic DNA is captured on a silicone membrane in a conventional spin column format. DNA is washed and then eluted from the spin column.

Epicentre Limited produces the SoilMaster DNA extraction kit which utilizes a hot detergent lysis process combined with a chromatographic step, which removes enzymatic inhibitors known to co-extract with DNA from soil and sediment samples.

Qbiogene Inc also produces a range of kits for extraction of DNA and RNA from soil examples. The kits are based on their FastPrep™ system.

### EXPERIMENTAL

### Soil Column Experiments

Three soil columns were set up and the following gases were introduced through the base of the column at 40 ml/min over a 17-day time period.

### Column 1—air

Column 2—a mix of butane, benzene, toluene, xylene and octane in air. Each component was present at 10 ppm.
Column 3—a mix of butane, benzene, toluene, xylene and octane in air. Each component was present at 100 ppm.

After 17 days, soil samples were collected at 2 cm, 4 cm, 7 cm and 9 cm above the base of the column. The DNA was extracted using one of the kits descried in the ‘Soil Sample Extraction Section’.

The modified EuBac assay was run on the soil samples to measure the copy number of the generic EuBac gene.

Quantitative PCR was used to obtain the copy number of the following indicator genes: 2,3CAT (Catechol 2-3 dioxygenase), a gene coding for an enzyme involved in aerobic aromatic degradation; AlkB-P1 a gene coding for an alkane dehydrogenase; and XyM a gene coding for xylene monooxygenase in the soil samples.

For every soil sample, the copy number of the indicator gene was divided by the EuBac copy number and expressed as a percentage. This gives the normalization index data (the ratio of the microbial population capable of metabolizing hydrocarbons to the overall microbial population). The resulting normalized data for 2,3CAT, AlkB-P1 and XyM is displayed graphically in FIGS. 4, 5 and 6 respectively.

The graphs show that in the soil exposed to hydrocarbons there is an enrichment of the indicator genes in the bacterial population in comparison to the air control. The results shown that the presence of hydrocarbons causes the relative number of copies of the hydrocarbon metabolizing genes to increase.

It is noted that in this case the enrichment of the indicator could be seen with the raw indicator gene copy number data alone. However, this is only because a proper control (soil exposed to air only) has been established. In practice, soil samples obtained from various geographical locations would not have a proper, identical control. Therefore normalization is important to provide an accurate indication of the presence of hydrocarbon deposits.

1. A method for detecting the presence of a hydrocarbon deposit in a geographical location comprising the steps of:
detecting the presence, at the location, of a target polynucleotide encoding a protein capable of metabolising a hydrocarbon, wherein the presence of the target polynucleotide is indicative of the presence of the hydrocarbon deposit;
determining the concentration of the target polynucleotide at the location; and
determining a value related to the concentration of bacteria at the location and calculating the ratio of the concentration of the target polynucleotide to the value.

2. A method according to claim 1 wherein the step of determining the value related to the concentration of bacteria at the location comprises the step of determining the concentration of a generic polynucleotide present in a plurality of different types of bacteria.

3. A method according to claim 1 wherein the step of detecting the presence of the target polynucleotide comprises detecting the presence of a subsequence of the target polynucleotide sequence.

4. A method according to claim 3 wherein the subsequence comprises a consensus sequence present in a plurality of different genes encoding a protein capable of metabolising a hydrocarbon.

5. A method according to claim 1 wherein the target polynucleotide is DNA.

6-17. (canceled)

18. A method according to claim 1 wherein the target polynucleotide is RNA.

19. A method according to claim 1 wherein the hydrocarbon is selected from the group consisting of: a C1 to C20 alkane, an alkene, an optionally substituted single or multi-ring aromatic hydrocarbon, and a naphthene.

20. A method according to claim 19 wherein the hydrocarbon is C2 to C20 alkane.

21. A method according to claim 1 wherein the protein capable of metabolising a hydrocarbon is selected from the group consisting of: a biphenyl dioxygenase, a toluene monooxygenase, an alkane hydroxylase; a catechol 2,3,dioxygenase; a naphthalene dioxygenase; a toluene dioxygenase; a xylene monooxygenase; a butane monooxygenase; a bacterial P450 oxygenase; or a eukaryotic P450 oxygenase.

22. A method according to claim 21 wherein the target polynucleotide is an alkB gene.

23. A method according to claim 1 wherein the presence of the target polynucleotide is determined using PCR.

24. A method according to claim 1 wherein concentration of the target polynucleotide is determined by quantitative PCR.

25. A method according to claim 1 wherein the concentration of the generic polynucleotide is determined by quantitative PCR.

26. A method according to claim 1 wherein the step of detecting the presence of the target polynucleotide comprises obtaining a soil sample from the location and detecting the presence of the target polynucleotide in the soil sample.

27. A method according to claim 26 wherein the soil sample is obtained from a depth of between 10 and 50 cm below the surface.

28. A method according to claim 26 further comprising the step of, after obtaining the soil sample, stabilising the nucleic acids in the soil sample.

29. A method according to claim 26 comprising the step of obtaining a plurality of soil samples at different sites at the geographical location and carrying out the method on each soil sample.

30. A method according to claim 29 and further comprising the step of correlating the results of the method on each soil sample, thereby determining the variation in the presence of the target polynucleotide at different sites within the geographical location.

31. A method according to claim 30 further comprising the steps of determining the concentration of the target polynucleotide and generating a first index related to said concentration at each site; determining the concentration of free hydrocarbon gas above each site and generating a second index related to said free hydrocarbon gas concentration at each site; and combining the first and second indexes for each site at the geographical location.
